(19) United States
(12) Patent Application Publication
(10) Pub. No.: US 2003/0004568 A1
(43) Pub. Date: Jan. 2, 2003

(54) Coated combination vaso-occlusive device

(75) Inventors: Christopher G.M. Ken, San Mateo, CA (US); Tina J. Patel, San Carlos, CA (US)

Correspondence Address: BANNER & WITCOFF, 1001 G STREET N W, SUITE 1100, WASHINGTON, DC, 20001, US

(73) Assignee: Concentric Medical, Mountain View, CA

(21) Appl. No.: 10/138,781

(22) Filed: May 6, 2002

Related U.S. Application Data
(60) Provisional application No. 60/288,467, filed on May 4, 2001.

(51) Int. Cl. A61F 2/06

(52) U.S. Cl. 623/1.46; 606/158

(57) ABSTRACT

Methods, compositions and apparatus are disclosed for treating abnormal conditions within a body. The apparatus includes vaso-occlusion devices each comprising a core formed of a metal, metal alloy, or non-metal material. Each core is coated with a polymer material that can include a bioactive agent. The methods include treating patients having abnormal blood flow at a site in their body by implanting such a coated vaso-occlusive device into the body at the site of the abnormal blood flow. The methods also include a method of making the vaso-occlusion devices. The composition includes a coating for the vaso-occlusive devices.

### CROSS-REFERENCE TO RELATED APPLICATION

This application claims benefit under 37 CFR § 1.78 of provisional application 60/288,467, filed May 4, 2001. The full disclosure of the application is incorporated herein by reference.

### FIELD OF THE INVENTION

The present invention relates to medical devices, methods and compositions for a coated vaso-occlusive device useful in treating conditions of abnormal blood flow in a patient.

### BACKGROUND OF THE INVENTION

Ruptured blood vessels in the brain cause an acute condition known as hemorrhagic stroke. Ruptures or strokes can occur with a number of vascular abnormalities including arterio venous malformation (AVM), fistula, aneurysm (a ballooning of the arterial wall), or a burst blood vessel. In addition, abnormal vasculature is generated in the process of tumor growth and tumors including brain tumors are highly vascularized entities requiring larger than normal blood flow to sustain the tumor.

Endovascular therapy for vaso-occlusion has included injectable agents, balloon-type occlusive devices, and mechanical vaso-occlusive devices such as metal coils. A description of these agents and devices is included in the background section of U.S. Pat. No. 4,994,069.

Currently, coils for aneurysms and polyvinyl alcohol (PVA) particles for AVMs are FDA approved preventative therapies. Cyanoacrylate glue for AVMs is also proposed and pending approval.

Over 400,000 persons worldwide, and 125,000 persons in the U.S. annually experience some form of hemorrhagic stroke or blood vessel rupture in the brain. Currently, a need exists in the medical community and the field of interventional neurology to expand and develop devices and/or agents for use in interventional neurology treatments for strokes and tumors.

A need also exists for a vaso-occlusive device that can be deployed at a site of abnormal blood flow and have an ability to create a natural biological response for either organized thrombi formation or endotheliazation to ensure total occlusion of the bleeding region.

### SUMMARY OF THE INVENTION

The invention provides a vaso-occlusive device for implantation into the vasculature of a patient to occlude abnormal blood flow therein comprising:

a member formed of a biocompatible material and coated with a composition comprising a polymer and a bioactive agent capable of reactivity at the site of implantation, wherein said member assumes a first, pre-implantation shape prior to being placed within said patient and a second, vaso-occlusive shape upon implantation in the patient, said first shape being different from said second shape.

The metal or metal alloy can be selected from the group consisting of platinum, stainless steel, nickel-titanium alloy, tungsten, gold, rhenium, palladium, rhodium, ruthenium, titanium, nickel, and alloys thereof.

If the material comprises a non-metal, the non-metal material can be a polymer or two or more polymers in a blend or copolymer.

The coating polymer can be selected from the group consisting of polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-phydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), polyglycolic lactic acid (PGLA), copolymers thereof, and blends of polymers thereof.

The coating polymer can be a natural polymer. The natural polymer can be selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin pectin, elastin, keratin, copolymers thereof, and blends of polymers thereof.

The bioactive agent can be selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The bioactive agent can be a tissue adhesion factor, and the tissue adhesion factor is selected from the group consisting of fibrin, collagen, albumin, cyanoacrylate, fibrinogen, chitosan, and gelatin-genipin.

The bioactive agent can be integrated into the coating polymer.

The coating polymer can be coated onto the metal or non-metal and the bioactive agent can be coated onto the polymer coating.

The invention includes a method of making a vaso-occlusive device comprising:

coating a member formed of a biocompatible material with a composition comprising a polymer and a bioactive agent, said member being formed of a material that assumes a pre-implantation shape prior to being introduced into a body and a vaso-occlusive shape when implanted with the body.

Coating can comprise spraying, dipping, jacketing, weaving, braiding, spinning, ion implantation, plasma deposition, and vapor deposition.

The metal or metal alloy material can be selected from the group consisting of platinum, stainless steel, nickel-titanium alloy, tungsten, gold, rhenium, palladium, rhodium, ruthenium, titanium, nickel, and alloys thereof.

The coating polymer can be selected from the group consisting of polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-phydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), polyglycolic lactic acid (PGLA), copolymers thereof, and blends of polymers thereof.

The polymer can be a natural polymer. The natural polymer can be selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin pectin, elastin, keratin, copolymers thereof, and blends of polymers thereof.

The bioactive agent can be selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The bioactive agent can be a tissue adhesion factor, and the tissue adhesion factor is selected from the group consisting of fibrin, collagen, albumin, cyanoacrylate, fibrinogen, chitosan, and gelatin-genipin.

The coating can comprise first coating the metal, metal alloy, or non-metal material with the coating polymer, and then coating or integrating a bioactive agent onto or into the coating polymer. The coating can also include the application of a composition that includes both the coating polymer and the bioactive agent. The coating can be accomplished during a process of implantation of the device in the patient.

The invention also includes a method of treating a patient having abnormal blood flow at a site, comprising:

providing a vaso-occlusive device comprising a biocompatible material coated with a composition comprising a polymer and a bioactive agent, said vaso-occlusive device having a pre-implantation shape;

implanting said vaso-occlusive device at the site;

changing the shape of said vaso-occlusive device from said pre-implantation shape to a vaso-occlusive shape; and

allowing the bioactive agent to react at said site.

The metal or metal alloy material can be selected from the group consisting of platinum, stainless steel, nickel-titanium alloy, tungsten, gold, rhenium, palladium, rhodium, ruthenium, titanium, nickel and alloys thereof.

Where the material comprises a non-metal and the non-metal material can be a polymer or two or more polymers in a blend or copolymer.

The coating polymer can be selected from the group consisting of polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-phydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), polyglycolic lactic acid (PGLA), copolymers thereof, and blends of polymers thereof.

The polymer can be a natural polymer. The natural polymer can be selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin pectin, elastin, keratin, copolymers thereof, and blends of polymers thereof.

The bioactive agent can be selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The bioactive agent can be a tissue adhesion factor, and the tissue adhesion factor is selected from the group consisting of fibrin, collagen, albumin, cyanoacrylate, fibrinogen, chitosan, and gelatin-genipin.

The bioactive agent can be integrated into the coating polymer. Where the coating polymer is coated onto the metal, metal alloy or non-metal material and the bioactive agent can be coated onto or integrated into the polymer coating.

The invention further provides a composition comprising an amount of a degradable or carrier polymer capable of being coated on a metal, metal alloy, or non-metal material and an effective amount of a bioactive agent integral to said degradable or carrier polymer for release at a site of implantation.

The degradable or carrier polymer can be selected from the group consisting of consisting of polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), polyglycolic lactic acid (PGLA), copolymers thereof, and blends of polymers thereof.

The degradable or carrier polymer can be a natural polymer and the natural polymer can be selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin pectin, elastin, keratin, copolymers thereof, and blends of polymers thereof.

The bioactive agent can be selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

### BRIEF DESCRIPTION OF THE DRAWINGS

FIG. 1A shows a vaso-occlusive device according to the present invention including a coated coil having a pre-implantation shape; and FIG. 1B shows the vaso-occlusive device of FIG. 1A with the coated coil in a vaso-occluding shape.

FIG. 2A depicts another embodiment of the vaso-occlusive device according to the present invention having a coil coated with a weave or braid; FIG. 2B shows yet another embodiment of the vaso-occlusive device according to the present invention having a coil coated with a weave with extending fibers.

### DETAILED DESCRIPTION

The following embodiments and examples are offered by way of illustration and not by way of limitation.

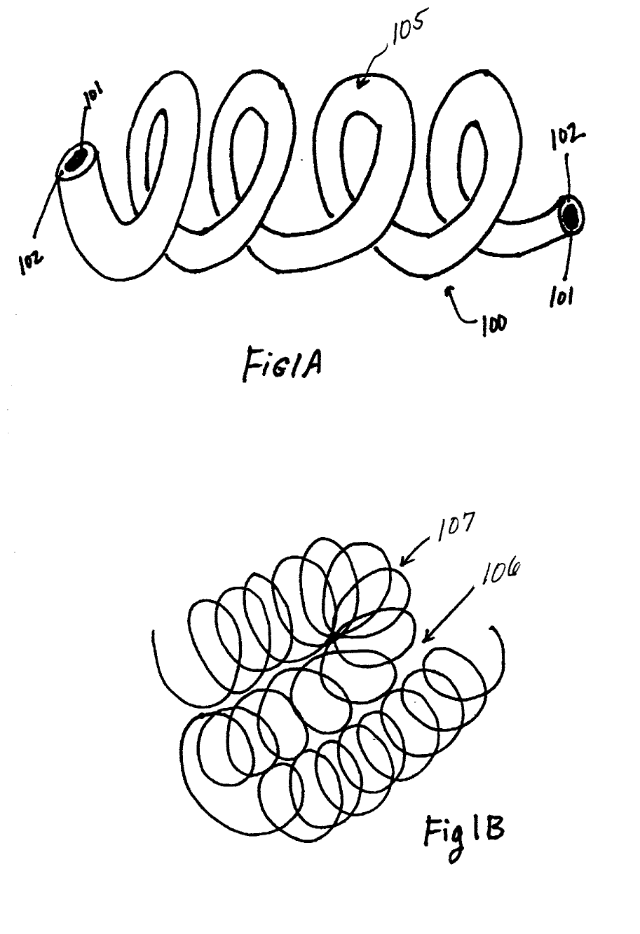

Turning first to the Figures, FIG. 1A depicts a vaso-occlusive device 100 according to the present invention. The device 100 includes a coil 105 with a core member (hereinafter “core”) 101 formed of a metal, metal alloy, or non-metal material and a biodegradable coating composition 102 surrounding at least a portion of the core 101. In a preferred embodiment, the biodegradable coating includes a polymer as discussed below. As shown in FIG. 1A, the device 100 can assume a pre-implantation shape that is substantially that of a helical coil. However, as readily understood, the device 100 can assume other known shapes prior to its deployment into the body. The term “shape” as used herein encompasses the terms “form,” “structure” and “configuration”.

FIG. 1B depicts the vaso-occlusive device 100 of FIG. 1A after implantation in a patient. The implanted device 100 assumes a vaso-occluding coiled coil or tangled coil 106 and 107 shape for occluding abnormal blood flow at a site of implantation. As shown in FIG. 1B, the tangled coil shape includes at least two spaced segments 106, 107 of the coil 105 that are entangled with each other.

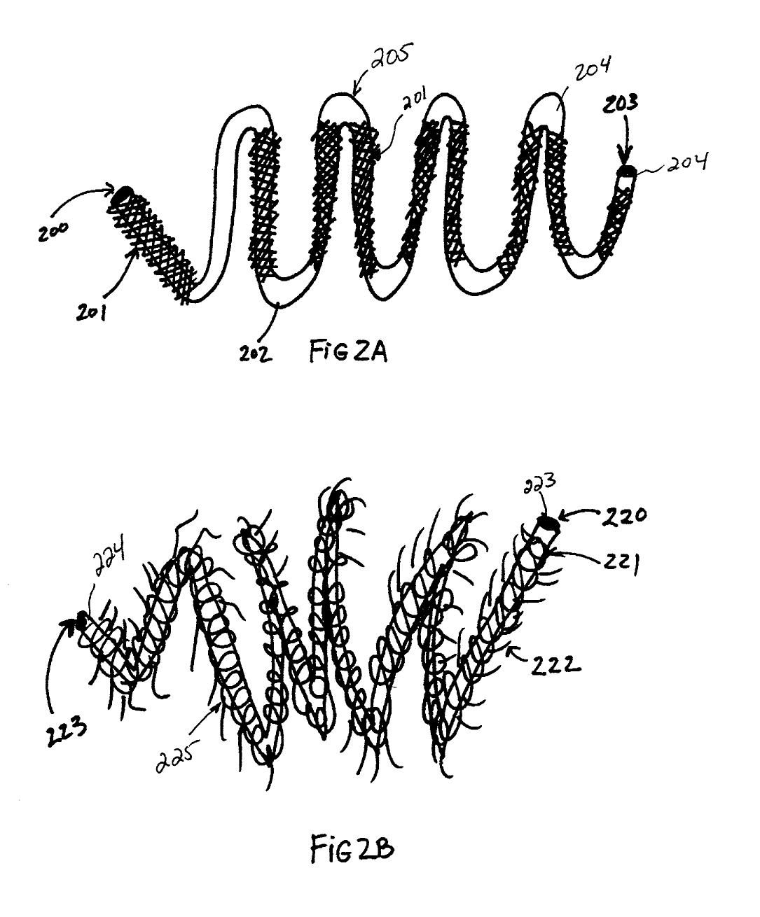

FIG. 2A depicts another embodiment of the vaso-occluding device 200 according to the present invention. Device 200 includes a coil 205 that is similar to coil 105. As shown, the coil 205 comprises a core member (hereinafter “core”) 203 formed of a metal, metal alloy or non-metal material and a biodegradable coating composition 204 that covers at least a portion of the core 203. The device 200 also includes a weave of material 201 that coats at least a portion of the coil 205 (FIG. 2A). In a preferred embodiment, portions 202 of the coil 205 are not coated with the weave of material. In an alternative embodiment, the weave of material 201 is applied directly to the core 203. The portions of the core 203 that are not covered by the weave of material 201 can be covered by the coating composition 204, left exposed or covered by a bioactive agent.

FIG. 2B depicts another embodiment of the vaso-occlusive device 220 according to the present invention. In this embodiment, the device 220 includes a coated coil 225 that is similar to coils 105 and 205. The coil 225 comprises a core member (hereinafter “core”) 223 formed of a metal, metal alloy or non-metal material and a biodegradable coating composition 224 that covers at least a portion of the core 223. At least a portion of the coil 225 is coated with strands of material 221. In the illustrated embodiment, substantially the entire coil 225 is wrapped with the strands of material 221. As shown in FIG. 2B, fibers 222 extend out from the wrap 221. In an alternative embodiment, the strands of material 221 are applied directly to the core 223. The portions of the core 223 that are not covered by the strands of material 221 can be covered by the coating composition 224, covered with a bioactive agent or left exposed.

The present invention includes a composition for coating the metallic or non-metallic cores 101, 203, 223 of the vaso-occlusive devices 100, 200, 220. The coating composition comprises a polymer and an effective amount of a bioactive agent. An effective amount will be determined in part by the level of activity desired after implantation of the device 100, 200, 220 and can be controlled by such factors as, for example the release rate of the bioactive agent, potency of the bioactive agent, and the desired effect.

In any of the above-discussed embodiments, the metallic cores 101, 203, 223 of the vaso-occlusive devices 100, 200, 220 can be made of any metal suitable for implantation into a patient, for example, but not limited to, e.g. platinum, stainless steel, nickel-titanium alloy, tungsten, gold, rhenium, palladium, rhodium, ruthenium, titanium, nickel, and alloys thereof. The non-metallic cores 101, 203, 223 of the vaso-occlusive devices 100, 200, 220 can be made of a polymer, copolymer or blend of polymers, using, for example, but not limited to, any of the polymers listed herein. No matter the embodiment, the cores 101, 203, 223 of the vaso-occlusive devices 100, 200, 220 are coated with the mixture of the coating polymer and the bioactive agent and upon implantation in a patient at a site of abnormal blood flow, abnormal blood flow is occluded.

The coating polymer can be biodegradable or may be a carrier polymer for the bioactive agent, and remain as a matrix or nondegrading structure around the vaso-occlusive device. If the coating polymer is biodegradable, as the polymer degrades in the body at the site of implantation, the bioactive agent is released at the site and it reacts or acts at the site according to the nature of the bioactive agent. The coating polymer can be any polymer-based molecule that can coat metal, metal alloy, or non-metal material. Suitable definitions for the terms biocompatible and biodegradable are found in Katz, Medical Devices and Diagnostic Industry, January 2001, “Developments in Medical Polymers for Biomaterials Applications”, pp 122-132. Materials for use in making the vaso-occlusive devices are also describe in Katz.

The coating polymer can be, e.g., selected from the group consisting of but not limited to polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), and Polyvinylpyrrolidone (PVP), polyglycolic lactic acid (PGLA), or similar compounds with similar qualities capable of coating metal, biodegrading and release a bioactive agent from the composition. The PGLA disclosed herein is formed by mixing PGA and PLA in ratios of 99.9:00.1 to 50:50.

The coating polymer may also be a natural polymer, e.g. polymers such as, but not limited to collagen, silk, fibrin, gelatin, hyaluron, cellulose, chitin, dextran, casein, albumin, ovalbumin, heparin sulfate, starch, agar, heparin, alginate, fibronectin, fibrin and pectin, elastin, keratin, copolymers thereof, and blended polymers thereof, and the like that can coat metal, metal alloy, or non-metal material and retain and release a bioactive agent as the coating polymer itself either degrades in the body or remains on the device as a matrix. In one embodiment, the coating could be a combination of natural and hydrogel copolymers.

As discussed above, the vaso-occlusive device 100, 200, 220 can also comprise a bioactive agent that is reactive at the site of implantation. For example, the bioactive agent may promote maintaining the device 100, 200, 220 at the site of abnormal blood flow, may promote regrowth of a damaged vascular wall, may help to heal the site, may inhibit continued or re-vascularization, may inhibit or regress tumor growth, and such like biological activities at the site of implantation or abnormal blood flow.

The bioactive agent can be any bioactive agent possessing a desired bioactivity. Thus, for example and not by way of limitation, bioactive agents can include a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue. The bioactive agent can be a tissue adhesion factor, and the tissue adhesion factor can be selected, for example, but not limited to, fibrin, collagen, albumin, cyanoacrylate, fibrinogen, chitosan, and gelatin-genipin.

In general, any agent having a desired bioactivity can be used in the coating. An effective amount of a bioactive agent is that amount of agent that will mix with the coating polymer to form the composition of the invention so that upon release of the bioactive agent at the site of implantation, the bioactivity generated promotes the desired change at the site. The effectiveness and thus the quantity of the bioactive agent needed will depend on the nature of the bioactive agent, its potency, the rate of release of the bioactive agent from the composition, and other such variable factors. The effective amount can be determined by standard assays known in the art for each bioactive agent selected. Some standard assays for bioactive agents are provided in books having standard laboratory procedures and assays. Additionally, more than one bioactive agent can be used in the composition, such as for example, a drug and an antibody.

The bioactive agent can be integrated into the coating polymer. The coating polymer can alternatively be coated onto the metal and the bioactive agent is coated onto the polymer coating. Coating can be accomplished, for example, by spraying, dipping, jacketing, weaving, braiding, spinning, ion implantation, plasma deposition, and vapor deposition. These coating processes may be accomplished as is standard in the art.

U.S. Pat. No. 5,808,012 describes a process usable with the present invention by which proteins and other bioactive agents can be incorporated into a polymer during a forming process such as extrusion, molding or casting, and which principles can also be applied to coating.

U.S. Pat. No. 6,184,348 describes production of novel polymers using recombinant techniques, and also integration of bioactive agents potentially useful at a site of implantation in the patient, providing an alternate means of coating or integrating the bioactive agent into the coating polymer. This production can be used with the present invention.

The cores 101, 203, 223 of the coated metal or non-metal vaso-occlusive devices 100, 200, 220 according to the present invention can assume a pre-implantation shape and an implanted or vaso-occluding shape. As a result, each coil 105, 205, 225 will also assume a pre-implantation and a vaso-occluding shape. When in its pre-implantation shape, the core 101, 203, 223 is coated with the composition comprising a polymer and a bioactive agent. The pre-implantation shaped device 100, 200, 220 is then implanted at the site of abnormal blood flow. During or after the introduction of the device 100, 200, 220 into the body, the device 100, 200, 220 assumes its vaso-occluding shape at a desired site, such as a site of abnormal blood flow. As shown in the figures, this vaso-occluding shape is different from the pre-implantation shape. For example, in its vaso-occluding shape, the portions 105, 106 of the device 100 can become entangled with each other (see FIG. 1B).

Pre-implantation shapes and vaso-occluding shapes can be any combination of shapes that are implantable (the pre-implantation shape) and that help to promote vaso-occlusion after implantation (the vaso-occluding shape). Thus for example, the pre-implantation shape can be (but is not limited to) a strip, rod, sheet, roll, tube, ribbon, string, and a coil. The vaso-occluding shape can comprise a shape including (but not limited to) for example a coil, a coiled coil, a circle, a half circle, a cone, a twisted sheet, a rod of random bends, and a helix.

Examples of permissible shapes for pre-implantation or vaso-occluding shapes of the device 100, 200, 220 include but are not limited to those shapes described in DES 407,818(spherical); knotted and tangling coil as described in Ritchart U.S. Pat. No. 4,994,069; a helical coil in a sinusoidal wave configuration, Chee U.S. Pat. No. 5,304,194; a vaso-occlusion braid of woven fibers, Engleson U.S. Pat. No. 5,423,849; a vaso-occlusive coil which is segmented onto which a fibrous woven or braided tubular covering or element is attached, Phelps U.S. Pat. No. 5,522,822; thrombogenic fibers in a central region containing a majority of these fibers upon ejection from the catheter, Mirigian U.S. Pat. No. 5,549,624; helically wound coil which helix is wound in such a way as to have multiple axially offset longitudinal or focal axes, Mariant U.S. Pat. No. 5,639,277; helical metallic coil having a plurality of axially spaced windings and a plurality of strands of a thrombogenic polymer extending axially through the central core of the coil, Snyder U.S. Pat. No. 5,658,308; proximal portion sufficiently flexible to fold on itself, Kupiecki U.S. Pat. No. 5,669,931; a vaso-occlusive helical metal coil having a thermoplastic polymer plug at one end or both, Gia U.S. Pat. No. 5,690,667; complex helically wound coil made up of pre-implantation helically wound coil which is wound in a vaso-occluding shape which is itself a series of helical turns, Wallace U.S. Pat. No. 5,733,329; a variable stiffness coil, Samson U.S. Pat. No. 5,766,160; a conical tipped cylindrical device with filamentary material, Wallace U.S. Pat. No. 5,957,948; helix in a tangled mass, Kupiecki U.S. Pat. No. 6,168,592; the shapes described in Berenstein et al, U.S. Pat. No. 5,826,587; the 3-dimensional in-filling vaso-occlusive coil of Mariant U.S. Pat. No. 5,957,948; the coil depicted in Engleson U.S. Pat. No. 6,024,754, and the multilayered vaso-occlusive coils of Ken et al, U.S. Pat. No. 6,033,423.

The change in-between the pre-implantation and vaso-occluding shapes in a given device 100, 200, 220 can be slight, and may result merely upon implantation and release from a container or implantation or delivery tool, and thus the vaso-occluding shape resulting from a given deliverable pre-implantation shape may be random and somewhat unpredictable. Alternatively, the change in shape can be due to the material alloy used for the core 101, 203, 223. In an embodiment in which the core 101, 203, 223 is formed of a shape-memory alloy such as Nitinol, the shape of the device 100, 200, 220 can change in response to the body temperature experienced after implantation.

The vaso-occlusive device 100, 200, 220 can also comprise a radio pacifier. The radio pacifier can comprise an agent that provides visibility of the device under X-ray or other imaging technology such as CT scans, MRIs and flouroscopy. The radio pacifier permits the device 100, 200, 220 to be monitored and detected once inside the patient. The radio pacifier can comprise, for example, a contrast media or a metal powder, but is not limited to these items. The metal powder can be, for example, titanium, tungsten, gold, bismuth, barium sulfate or tantalum powder. Additionally, the radio pacifier includes a gadolinium-based MRI contrast agent. These agents can include, but are not limited to, Gadopentetate, Gadopentetate dimeglumine (Gd DTPA or Magnevist (R)), Gadoteridol (Gd HP-DO3A or ProHance (R)), Gadodiamide (Gd DTPA-BMA or Omniscan (R)), Gadoversetamide (Gd DTPA-BMEA or OptiMARK (R)), Gd-DOTA (Magnevist (R) or Dotarem (R)), Gd-DTPA labeled albumin, and Gd-DTPA labeled dextran.

In an embodiment, the coils 105, 205, 225 are delivered to the surgeon, other practitioner or attendant in pre-cut or pre-formed lengths. In this embodiment, each coil is cut to a predetermined length. For example, the length of the coils 105, 205, 225 of the vaso-occlusive device 100, 200, 220 as it is delivered can be in the range from about 1 mm to about 5 meters. In a preferred embodiment, the lengths of the coils 105, 205, 225 of the vaso-occlusive device 100, 200, 220 for delivery to the patient can be in a range from about 1 mm to about 10 mm. In an embodiment, the dimensions of the device 100, 200, 220 can be from about 0.125 mm to about 12.50 mm, or the outside diameter of objects suitable for passing through a delivery device to a site of abnormal bleeding. The diameter of the vaso-occlusive device 100, 200, 220 once it is delivered and after it has assumed its vaso-occluding shape (FIG. 1B) can be in a range from about 1 mm to about 50 mm.

Dimensions for the vaso-occlusive device 100, 200, 220 for delivery to the patient can be in a range from about 0.005 inches to about 0.50 inches, or the outside diameter of objects suitable for passing through a delivery device to a site of abnormal bleeding. The lengths of the vaso-occlusive device 100, 200, 220 as it is delivered can comprise in the range from about 1 mm to about 5 meters. The diameter of the vaso-occlusive device 100, 200, 220 once it is delivered and after it has assumed its vaso-occluding shape can be in a range from about 1 mm to about 50 mm.

The present invention also includes a method of making the coated vaso-occlusive devices 100, 200, 220 described above. The method comprises coating a metal, metal alloy, or non-metal material having a pre-implantation shape (such as those shapes described herein) with at least one of the compositions described above, or a coating that includes either one of the above-discussed polymers or bioactive agents, such that the coating still allows the pre-implantation shape to form the vaso-occlusive shape during or after implantation in the patient. The change from the pre-implantation shape to the vaso-occluding shape can be slight. As discussed above, this change is shape can result from a mere release of the device 100, 200, 220 in an implantation delivery device to the site where the vaso-occlusive device 100, 200, 220 conforms to the space differential (between the delivery device and the site of abnormal blood flow) or in response to the body heat experienced by a shape memory alloy from which the device is formed.

Coating can be accomplished by any method or process that effectively provides a layer of the composition on at least part of the metal coil core 101, 203, 223 of the vaso-occluding device 100, 200, 220. Thus, for example, but not by way of limitation, the composition can be contacted with the metal or alloy core 101, 203, 223 by spraying, dipping, jacketing, weaving, braiding, spinning, ion implantation, plasma deposition, and vapor deposition. Weaving, braiding and spinning would entail taking threads of the material and winding, weaving, braiding or sewing them in and around the metal, metal alloy, or non-metal material of the core 101, 203, 223. The presumption here is that the core 101, 203, 223 will be effectively coated by such efforts to surround the metal or non-metal material with strands of the composition. Coating can be accomplished generally as described in Odowaki et al, 2000 Society for Biomaterials “Development of Argatoban Coated Metallic Stent for Prevention of Post-Operative Restenosis”, pp. 1023, 6^{th} World Biomaterials Congress transactions. Spinning is described in U.S. Pat. No. 6,184,348.

Coating can be accomplished in a process of implanting or delivering the device into the patient, e.g. the metallic or non-metallic core 101, 203, 223 of the device 100, 200, 223 is loaded in a solution of a polymer and a bioactive agent, and upon release in the patient, the coating solution coats the core 101, 203, 223 and the coated device 100, 200, 220 is released into the patient, as a coated article (thereafter assuming a vaso-occlusive shape).

The invention also provides a method of treating a patient having abnormal blood flow at a site in the body. The method comprises implanting into the patient the coated vaso-occlusive device 100, 200, 220 of the invention as described herein. The core 101, 203, 223 of the device 100, 200, 220 is coated with at least one of the compositions discussed above comprising a polymer and bioactive agent. Once coated, the pre-implantation shaped device 100, 200, 220 is implanted into the patient and the device assumes its vaso-occluding vaso-occluding shape as described above.

The vaso-occlusive device 100, 200, 220 is especially useful for treating vessel ruptures, aneurysms, AVMs, fistulas, benign or malignant tumors and other conditions manifesting abnormal blood flow.

All publications, patents and patent applications cited in this specification are herein incorporated by reference as if each individual publication, patent or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is

1. A vaso-occlusive device for implantation into the vasculature of a patient to occlude abnormal blood flow therein comprising:
a member formed of a biocompatible material and coated with a composition comprising a polymer and a bioactive agent capable of reactivity at the site of implantation, wherein said member assumes a first, pre-implantation shape prior to being placed within said patient and a second, vaso-occlusive shape upon implantation in the patient, said first shape being different from said second shape.

2. A vaso-occlusive device as in claim 1, wherein the biocompatible material comprises a metal or metal alloy selected from the group consisting of platinum, stainless steel, nickel-titanium alloy, tungsten, gold, rhenium, palladium, rhodium, ruthenium, titanium, nickel, and alloys thereof.

3. A vaso-occlusive device as in claim 1, wherein the biocompatible material comprises a non-metal and the non-metal material is covered by at least one polymer, at least one copolymer or two or more polymers in a blend.

4. A vaso-occlusive device as in claim 1, wherein the coating polymer is selected from the group consisting of polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), polyglycolic lactic acid (PGLA), copolymers thereof, and blends of polymers thereof.

5. A vaso-occlusive device as in claim 1, wherein the coating polymer is a natural polymer.

6. A vaso-occlusive device as in claim 5, wherein the natural polymer is selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin pectin, elastin, keratin, copolymers thereof, and blends of polymers thereof.

7. A vaso-occlusive device as in claim 1, wherein the bioactive agent is selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

8. A vaso-occlusive device as in claim 7, wherein the bioactive agent is a tissue adhesion factor, and the tissue adhesion factor is selected from the group consisting of fibrin, collagen, albumin, cyanoacrylate, fibrinogen, chitosan, and gelatin-genipin.

9. A vaso-occlusive device as in claim 1, wherein the bioactive agent is integrated into the coating polymer.

10. A vaso-occlusive device as in claim 1, wherein the coating polymer is coated onto the metal or non-metal and the bioactive agent is coated onto the polymer coating.

11. A method of making a vaso-occlusive device comprising:
coating a member formed of a biocompatible material with a composition comprising a polymer and a bioactive agent, said member being formed of a material that assumes a pre-implantation shape prior to being introduced into a body and a vaso-occlusive shape when implanted with the body.

12. A method of making a vaso-occlusive device as in claim 11, wherein the coating step comprises spraying, dipping, jacketing, weaving, braiding, spinning, ion implantation, plasma deposition, and vapor deposition.

13. A method of making a vaso-occlusive device as in claim 11, wherein the biocompatible material comprises a metal or metal alloy material selected from the group consisting of platinum, stainless steel, nickel-titanium alloy, tungsten, gold, rhenium, palladium, rhodium, ruthenium, titanium, nickel, and alloys thereof.

14. A method of making a vaso-occlusive device as in claim 10, wherein the coating polymer is selected from the group consisting of polyacrylamide (PAAM), poly (N-isopropylacrylamine) (PNiPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(gethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), polyglycolic lactic acid (PGLA), copolymers thereof, and blends of polymers thereof.

15. A method of making a vaso-occlusive device as in claim 11, wherein the polymer is a natural polymer.

16. A method of making a vaso-occlusive device as in claim 15, wherein the natural polymer is selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin pectin, elastin, keratin, copolymers thereof, and blends of polymers thereof.

17. A method of making a vaso-occlusive device as in claim 11, wherein the bioactive agent is selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

18. A method of making a vaso-occlusive device as in claim 17, wherein the bioactive agent is a tissue adhesion factor, and the tissue adhesion factor is selected from the group consisting of fibrin, collagen, albumin, cyanoacrylate, fibrinogen, chitosan, and gelatin-genipin.

19. A method of making a vaso-occlusive device as in claim 11, wherein coating comprises first coating the biocompatible material with the coating polymer, and then coating or integrating a bioactive agent onto or into the coating polymer.

20. A method as in claim 11, wherein the coating is accomplished during a process of implantation of the device in the body.

21. A method of treating a patient having abnormal blood flow at a site, comprising:
providing a vaso-occlusive device comprising a biocompatible material coated with a composition comprising a polymer and a bioactive agent, said vaso-occliusive device having a pre-implantation shape;
implanting said vaso-occlus ive device at the site;
changing the shape of said vaso-occlusive device from said pre-implantation shape to a vaso-occlusive shape; and
allowing the bioactive agent to react at said site.

22. A method of treating a patient as in claim 21, wherein the biocompatible material comprises a metal or metal alloy material selected from the group consisting of platinum, stainless steel, nickel-titanium alloy, tungsten, gold, rhenium, palladium, rhodium, ruthenium, titanium, nickel and alloys thereof.

23. A method of treating a patient as in claim 21, wherein the biocompatible material comprises a non-metal and the non-metal material is a polymer or two or more polymers in a blend or copolymer.

24. A method of treating a patient as in claim 21, wherein the coating polymer is selected from the group consisting of polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), polyglycolic lactic acid (PGLA), copolymers thereof, and blends of polymers thereof.

25. A method of treating a patient as in claim 21, wherein the polymer is a natural polymer.

26. A method of treating a patient as in claim 25, wherein the natural polymer is selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin pectin, elastin, keratin, copolymers thereof, and blends of polymers thereof.

27. A method of treating a patient as in claim 21, wherein the bioactive agent is selected from the group consisting of a protein factor, a growth factor, an inhibiting factor, an endothelization factor, an extracellular matrix-forming factor, a cell adhesion factor, a tissue adhesion factor, an irmmunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

28. A method of treating a patient as in claim 27, wherein the bioactive agent is a tissue adhesion factor, and the tissue adhesion factor is selected from the group consisting of fibrin, collagen, albumin, cyanoacrylate, fibrinogen, chitosan, and gelatin-genipin.

29. A method of treating a patient as in claim 21, wherein the bioactive agent is int egrated into the coating polymer.

30. A method of treating a patient as in claim 21, wherein the coating polymer is coated onto the biocompatible material and the bioactive agent is coated onto or integrated into the polymer coating.

31. A composition comprising a degradable or carrier polymer capable of being coated on a metal, metal alloy, or non-metal material and a bioactive agent integral to said degradable or carrier polymer for release at a site of implantation.

32. A composition as in claim 31, wherein the degradable or carrier polymer is selected from the group consisting of consisting of polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(gethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), polyglycolic lactic acid (PGLA), copolymers thereof, and blends of polymers thereof.

33. A composition as in claim 31, wherein the degradable or carrier polymer is a natural polymer and the natural polymer is selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin pectin, elastin, keratin, copolymers thereof, and blends of polymers thereof.

34. A composition as in claim 31, wherein the bioactive agent is selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.
